SEQUENCE LISTING (14 sequences)

SEQ ID NO: 1 - DNA, 566 bp, artificial
  circularly permuted growth hormone
atgagccccggactgggcagatcttcaagcagacctacagcaagttcgacacaaactcacacaacgatgacgcactactcaagaactacgggctgctctactgcttcaggaaggacatggacaaggtcgagacattcctgcgatcgtgcagtgccgctctgtggagggcagcaccattcccttatccaggctttttgacaacgctagtctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaagggcatccaaacgctgatggggaggctggaagatctcgagtga

SEQ ID NO: 2 - protein, 188 aa, Artificial
  circular permuted growth hormone
MSPRTGQIFKQTYSKFDTNSHNDDALLKNYGLLYCFRKDMDKVETFLRIVQCRSVEGSTIPLSRLFDNASLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDLE

SEQ ID NO: 3 - protein, 5 aa, Artificial
  linker peptide
GGGGS

SEQ ID NO: 4 - protein, 246 aa, Homo sapiens
FSGSEATAAILSRAPWSLQSVNPGLKTNSSKEPKFTKCRSPERETFSCHWTDEVHHGTKNLGPIQLFYTRRNTQEWTQEWKECPDYVSAGENSCYFNSSFTSIWIPYCIKLTSNGGTVDEKCFSVDEIVQPDPPIALNWTLLNVSLTGIHADIQVRWEAPRNADIQKGWMVLEYELQYKEVNETKWKMMDPILTTSVPVYSLKVDKEYEVRVRSKQRNSGNYGEFSEVLYVTLPQMSQFTCEEDFY

SEQ ID NO: 5 - protein, 126 aa, Homo sapiens
FSGSEATAAILSRAPWSLQSVNPGLKTNSSKEPKFTKCRSPERETFSCHWTDEVHHGTKNLGPIQLFYTRRNTQEWTQEWKECPDYVSAGENSCYFNSSFTSIWIPYCIKLTSNGGTVDEKCFSVD

SEQ ID NO: 6 - protein, 120 aa, Homo sapiens
EIVQPDPPIALNWTLLNVSLTGIHADIQVRWEAPRNADIQKGWMVLEYELQYKEVNETKWKMMDPILTTSVPVYSLKVDKEYEVRVRSKQRNSGNYGEFSEVLYVTLPQMSQFTCEEDFY

SEQ ID NO: 7 - protein, 199 aa, Homo sapiens
LPICPGGAARCQVTLRDLFDRAVVLSHYIHNLSSEMFSEFDKRYTHGRGFITKAINSCHTSSLATPEDKEQAQQMNQKDFLSLIVSILRSWNEPLYHLVTEVRGMQEAPEAILSKAVEIEEQTKRLLEGMELIVSQVHPETKENEIYPVWSGLPSLQMADEESRLSAYYNLLHCLRRDSHKIDNYLKLLKCRIIHNNNC

SEQ ID NO: 8 - protein, 195 aa, artificial
  circular permuted growth hormone his tagged
HMSPRTGQIFKQTYSKFDTNSHNDDALLKNYGLLYCFRKDMDKVETFLRIVQCRSVEGSTIPLSRLFDNASLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDLEHHHHHH

SEQ ID NO: 9 - protein, 188 aa, artificial
  circular permuted growth hormone
MSPRTGQIFKQTYSKFDTNSHNDDALLKNYGLLYCFNADMSRVSTFLRTVQCRSVEGSTIPLSRLFDNASLRADRLNQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEERIQTLMGRLEDLE

SEQ ID NO: 10 - DNA, 588 bp, Artificial
  circular permuted growth hormone
catatgagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagcaccattcccttatccaggctttttgacaacgctagtctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaacgcatccaaacgctgatggggaggctggaagatctcgagcaccaccaccaccaccactga

SEQ ID NO: 11 - DNA, 567 bp, Artificial
  circular permuted growth hormone
atgagcccccggactgggcagatcttcaagcagacctacagcaagttcgacacaaactcacacaacgatgacgcactactcaagaactacgggctgctctactgcttcaacgccgacatgtcaagggtctcaacattcctgcgcacagtgcagtgccgctctgtggagggcagcaccattcccttatccaggctttttgacaacgctagtctccgcgccgaccgtctgaaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaacgcatccaaacgctgatggggaggctggaagatctcgagtga

SEQ ID NO: 12 - DNA, 32 bp, artificial
  PCR forward primer
tggataagggaatggtgctgccctccacagag

SEQ ID NO: 13 - DNA, 27 bp, artificial
  PCR primer
aattaattcatatgagcccccggactg

SEQ ID NO: 14 - DNA, 29 bp, artificial
  PCR primer
aattctcgagatcttccagcctccccatc

(19) United States
(12) Patent Application Publication
(10) Pub. No.: US 2010/0035804 A1
(43) Pub. Date: Feb. 11, 2010

(54) POLYPEPTIDE ANTAGONIST

(75) Inventors: Sarbendra Pradhananga, Sheffield (GB); John Sayers, Sheffield (GB); Richard Ross, Sheffield (GB); Peter Artymiuk, Sheffield (GB)

Correspondence Address: SPECKMAN LAW GROUP PLLC, 1201 THIRD AVENUE, SUITE 330, SEATTLE, WA, 98101, US

(73) Assignee: ASTERION LIMITED, Sheffield (GB)

(21) Appl. No.: 12/296,180

(22) PCT Filed: Apr. 5, 2007
(86) PCT No.: PCT/GB2007/001285

§ 371 Date: Jun. 16, 2009

(30) Foreign Application Priority Data
Apr. 6, 2006 (GB) 0606946.2

(51) Int. Cl. A61K 38/17; C12P 21/06; C12N 1/21; C12N 15/63; A61K 31/7088; C07K 14/435; C07H 21/04

(52) U.S. Cl. 514/12; 435/69.1; 435/252.33; 435/320.1; 514/44 R; 530/350; 536/23.4; 536/23.5

(57) ABSTRACT

We describe a circularly permuted growth hormone polypeptide antagonist; compositions comprising said antagonist and methods to treat conditions that would benefit from administration of said antagonist.

The invention relates to a circularly permuted growth hormone polypeptide antagonist; compositions comprising said antagonist and methods to treat conditions that would benefit from administration of said antagonist.

A large group of growth factors, referred to as cytokines, are involved in a number of diverse cellular functions. These include modulation of the immune system, regulation of energy metabolism and control of growth and development. Cytokines mediate their effects via receptors expressed at the cell surface on target cells. Cytokine receptors can be divided into four separate sub groups. Type 1 (growth hormone (GH) family) receptors are characterised by four conserved cysteine residues in the amino terminal part of their extracellular domain and the presence of a conserved Trp-Ser-Xaa-Trp-Ser motif in the C-terminal part. The repeated Cys motif is also present in Type 2 (interferon family) and Type III (tumour necrosis factor family).

It is known that many cytokine ligands interact with their cognate receptor via specific sites. Some cytokine receptors have both high affinity ligand binding sites and low affinity binding sites.

For example, it is known that a single molecule of GH associates with two receptor molecules (GHR) (Cunningham et al., 1991; de Vos et al., 1992; Sundstrom et al., 1996; Clackson et al., 1998). This occurs through two unique receptor-binding sites on GH and a common binding pocket on the extracellular domain of two receptors. Site 1 on the GH molecule has a higher affinity than site 2, and receptor dimerization is thought to occur sequentially with one receptor binding to site 1 on GH followed by recruitment of a second receptor to site 2. The extracellular domain of the GHR exists as two linked domains each of approximately 100 amino acids. It is a conformational change in these two domains that occurs on hormone binding with the formation of the trimeric complex GHR-GH-GHR. Internalisation of the GHR-GH-GHR complex is followed by a recycling step whereby the receptor molecule is regenerated for further use within the cell.

A variety of different stoichiometries are employed by different cytokines and other ligands on receptor binding. Thus erythropoetin, like GH, forms a trimeric receptor-hormone-receptor complex. Interleukin-4 forms a trimeric receptor-hormone-different receptor complex. Other cytokines, for example leptin and GCSF, form tetrameric receptor-hormone-hormone-receptor complexes, and others (eg interleukin 6) probably form hexameric complexes consisting of two soluble receptor molecules, two transmembrane receptor molecules and two cytokine molecules. In each case there is a primary high affinity binding site that locates the cytokine to the receptor complex, and additional sites which play secondary roles in altering the conformation or recruiting other molecules and thereby initiating signalling.

Variant cytokine polypeptides are known. For example, GH variants are disclosed in U.S. Pat. No. 5,849,535. The modification to GH is at both site 1 and site 2 binding sites. The modifications to site 1 produce a GH molecule that has a higher affinity for GHR compared to wild-type GH. These modified GH molecules act as agonists. There is also disclosure of site 2 modifications that result in the creation of GH antagonists. Further examples of modifications to GH which alter the binding affinity of GH for site 1 are disclosed in U.S. Pat. No. 5,854,026; U.S. Pat. No. 6,004,931; U.S. Pat. No. 6,022,711; U.S. Pat. No. 6,057,292; and U.S. Pat. No. 6,136,563. These modifications relate to point mutations at specific positions in GH which produce a molecule with altered signalling properties.

Circular permutation is a means to generate polypeptide variants that retain the overall linear primary sequence structure of a native polypeptide but re-orders the sequence by forming new amino and carboxyl termini. The process generates molecules with altered biological properties. The process includes the fusion of the natural amino and carboxyl termini either directly or by using linker molecules that are typically peptide linkers. The circularised molecule is then conceptually cut to create new amino and carboxyl termini. Circularly permuted polypeptides can be generated either recombinantly or by in vitro peptide synthesis.

Circular permutation has been used to generate chimeric molecules with altered biological activity.

For instance, WO95/27732 discloses the creation of a circularly permuted IL-4 ligand fused to a cytotoxic agent. The permuted IL-4-agent has altered affinity and cytotoxicity when compared to a native IL-4-agent and has efficacy with respect to killing cancer cells which are exposed to the conjugated polypeptide.

WO99/51632 describes the use of circular permutation to generate novel streptavidin binding proteins that have reduced affinity for biotin. The circularly permuted streptavidin is fused to a second polypeptide to create a fusion protein that differentially binds biotin. The reduced affinity of the strepavidin fusion protein for biotin facilitates release of the fusion protein when biotin is used as a drug delivery vehicle.

WO01/51629 discloses circularly permuted bacterial β-lactamase and its use as a marker protein for the detection of interactions between intracellular and extracellular proteins which assemble with the permuted polypeptide.

Methods to identify circularly permuted polypeptides are also known. For example, WO00/18905, which is incorporated by reference in its entirety, describes a method to identify permuted polypeptides, referred to as “permuteins”, using a phage display vector into which a library of permuted genes is inserted. The expression of the library at the surface of the display vector is detected by exposure of the expressed library to a binding protein which potentially interacts with a permutein.

WO01/30998, which is incorporated by reference in its entirety, discloses a further method to generate and identify circularly permuted proteins. The invention relates to the formation of fusion proteins comprising the amino terminal part of a first protein fused to the carboxyl terminal part of a different second protein from which permutations are synthesised. A library of fusion proteins is created which can be screened by phage display.

In our co-pending application WO 2005/003165A2 we disclose, amongst other things, circularly permuted growth hormone molecules. We disclose the agonist activity of one such molecule and the modification of this molecule to an antagonist of growth hormone receptor activity.

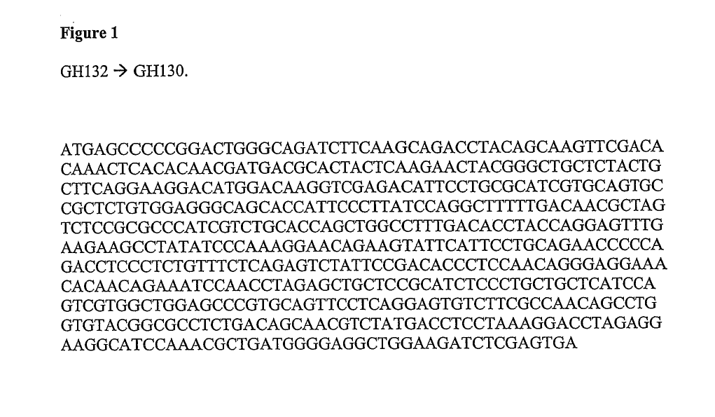

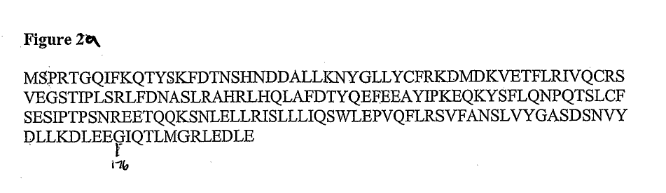

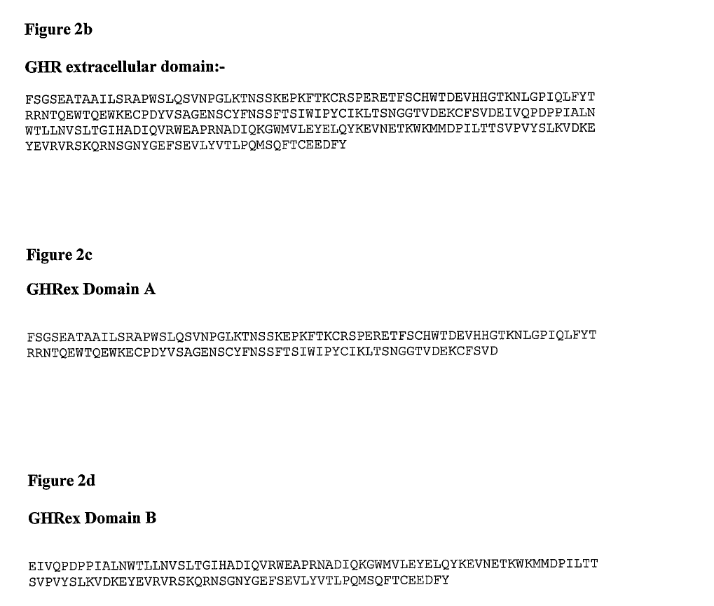

According to an aspect of the invention there is provided an isolated nucleic acid molecule comprising a nucleic acid sequence wherein said nucleic acid sequence is selected from the group consisting of:
(i) a nucleic acid molecule consisting of the sequence as represented in FIG. 1 (SEQ ID NO: 1);
(ii) a nucleic acid molecule comprising a sequence that hybridises to the sequence identified in (i) wherein said nucleic acid molecule includes a modification comprising a sequence that encodes amino acid residue 176 as indicated in FIG. 1, wherein said modification results in the addition, substitution or deletion of at least one amino acid residue and said nucleic acid molecule encodes a polypeptide with growth hormone receptor antagonist activity;
(iii) a nucleic acid molecule that encodes a polypeptide comprising an amino acid sequence as represented in FIG. 2a (SEQ ID NO: 2).

In a preferred embodiment of the invention there is provided an isolated nucleic acid molecule that anneals under stringent hybridisation conditions to the sequences described in (i) and (ii) above.

Hybridization of a nucleic acid molecule occurs when two complementary nucleic acid molecules undergo an amount of hydrogen bonding to each other. The stringency of hybridization can vary according to the environmental conditions surrounding the nucleic acids, the nature of the hybridization method, and the composition and length of the nucleic acid molecules used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, N.Y., 2001); and Tijssen, Laboratory Techniques in Biochemistry and Molecular Biology-Hybridization with Nucleic Acid Probes Part I, Chapter 2 (Elsevier, N.Y., 1993). The Tₘ is the temperature at which 50% of a given strand of a nucleic acid molecule is hybridized to its complementary strand. The following is an exemplary set of hybridization conditions and is not limiting:

Very High Stringency (Allows Sequences that Share at Least 90% Identity to Hybridize)

| | |
|---|---|
| Hybridization: | 5x SSC at 65° C. for 16 hours |
| Wash twice: | 2x SSC at room temperature (RT) for 15 minutes each |
| Wash twice: | 0.5x SSC at 65° C. for 20 minutes each |

High Stringency (Allows Sequences that Share at Least 80% Identity to Hybridize)

| | |
|---|---|
| Hybridization: | 5x-6x SSC at 65° C.-70° C. for 16-20 hours |
| Wash twice: | 2x SSC at RT for 5-20 minutes each |
| Wash twice: | 1x SSC at 55° C.-70° C. for 30 minutes each |

Low Stringency (Allows Sequences that Share at Least 50% Identity to Hybridize)

| | |
|---|---|
| Hybridization: | 6x SSC at RT to 55° C. for 16-20 hours |
| Wash at least twice: | 2x-3x SSC at RT to 55° C. for 20-30 minutes each. |

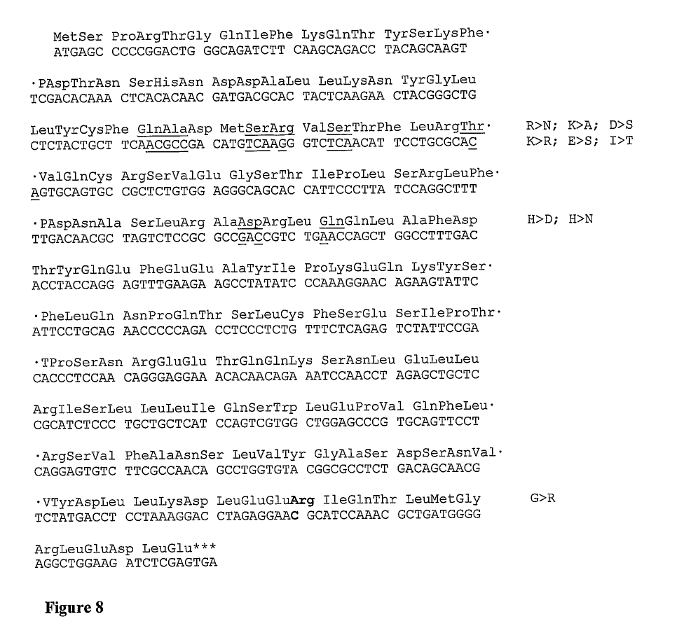

In a preferred embodiment of the invention said nucleic acid molecule encodes a polypeptide comprising an amino acid sequence as represented in FIG. 8 (SEQ ID NO: 9).

According to a further aspect of the invention there is provided a polypeptide comprising the amino acid sequence represented in FIG. 2 (SEQ ID NO: 2), which sequence has been modified by addition, deletion or substitution of at least one amino acid residue wherein said modification includes amino acid residue 176 wherein said polypeptide is a growth hormone receptor antagonist.

The polypeptide of the invention may differ in amino acid sequence by one or more substitutions, additions, deletions, truncations that may be present in any combination that includes amino acid residue 176.

In a preferred embodiment of the invention said polypeptide is modified by substitution of glycine at position 176 for an amino acid selected from the group consisting of: histidine, aspartic acid, valine, arginine, alanine, lysine, tryptophan, tyrosine, phenylalanine and glutamic acid.

Preferably said substitution is glycine 176 for arginine or lysine or alanine; preferably said modification is glycine for arginine.

In a preferred embodiment of the invention said polypeptide comprises an amino acid sequence as represented in FIG. 8 (SEQ ID NO: 9)

In addition, the invention features polypeptide sequences having at least 75% identity with the polypeptide sequences as herein disclosed, or fragments and functionally equivalent polypeptides thereof. In one embodiment, the polypeptides have at least 85% identity, more preferably at least 90% identity, even more preferably at least 95% identity, still more preferably at least 97% identity, and most preferably at least 99% identity with the amino acid sequences illustrated herein.

In a further embodiment of the invention there is provided a polypeptide according to the invention linked to at least one extracellular binding domain of growth hormone receptor to form a fusion protein; preferably said binding domain consists of the extracellular domain of growth hormone receptor.

In a preferred embodiment of the invention said domains are linked via a peptide linking molecule.

In a preferred embodiment of the invention said peptide linking molecule is a flexible peptide linker.

Preferably the linker is a peptide which comprises 5 to 30 amino acid residues. More preferably the linker comprises 10 to 20 amino acid residues.

More preferably the linker comprises at least one copy of the peptide:

(SEQ ID NO: 3)

Gly-Gly-Gly-Gly-Ser (referred to as “Gly4Ser”).

In one embodiment of the invention the linker is 10 amino acids in length and comprises two copies of the Gly4Ser linker. In an alternative embodiment of the invention, the linker is 15 amino acids in length and comprises three copies of the Gly4Ser linker. In yet an alternative embodiment, the linker is 20 amino acids in length and comprises four copies of the Gly4Ser linker.

In our co-pending application, WO01/096565, which is incorporated by reference in its entirety, we disclose fusion proteins which translationally fuse the ligand binding domain of a cytokine to the extracellular receptor binding domain of said ligand via peptide linkers. These fusion proteins have delayed clearance and agonist activity. Peptide linkers which link the polypeptide of the invention to one another to form oligomeric polypeptides (dimers, trimers etc) and to growth hormone extracellular receptor binding domains are either flexible or inflexible (e.g. helical) or of intermediate flexibility (e.g. a combinational linker which is part helical) as described in our co-pending application WO 2006/010891, which is incorporated by reference in its entirety. Linkers may also contain cleavage sites, for example protease cleavage sites to provide fusion polypeptides with delayed release characteristics; these are described in our co-pending application WO 03/062276 which is incorporated by reference in its entirety.

According to a further aspect of the invention there is provided a fusion polypeptide comprising at least two polypeptides according to the invention linked in tandem.

In a preferred embodiment of the invention there is provided a fusion polypeptide comprising a plurality of polypeptides according to the invention.

In a further preferred embodiment of the invention there is provided a fusion polypeptide consisting of two polypeptides according to the invention linked in tandem.

In an alternative preferred embodiment of the invention there is provided a fusion polypeptide comprising 3, 4, 5, 6, 7, 8, 9, 10 polypeptides according to the invention.

In a yet further preferred embodiment of the invention said fusion polypeptide comprising two or at least two polypeptides according to the invention that are linked together by a linker molecule. Preferably said linker molecule is as hereinbefore disclosed.

According to a yet further aspect of the invention there is provided a fusion polypeptide comprising at least two polypeptides according to the invention further comprising at least one growth hormone binding domain of a growth hormone receptor.

Preferably said fusion polypeptide consists of two polypeptides according to the invention and one growth hormone binding domain of a growth hormone receptor.

In a preferred embodiment of the invention said binding domain comprises an extracellular binding domain of growth hormone receptor; preferably said domain consists of the extracellular domain of growth hormone receptor.

According to a further aspect of the invention there is provided a chimeric fusion polypeptide comprising a polypeptide according to the invention linked, either directly or indirectly, to a prolactin polypeptide.

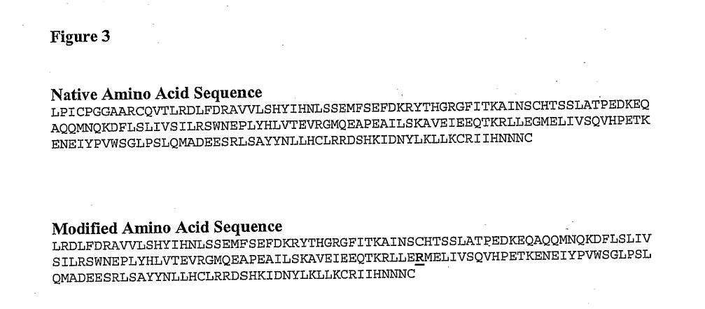

In a preferred embodiment of the invention said prolactin polypeptide comprises an amino acid sequence wherein said amino acid sequence is modified at position 129 of human prolactin as represented in FIG. 3 (SEQ ID NO: 7).

In a preferred embodiment of the invention said modification at position 129 as represented in FIG. 3 (SEQ ID NO: 7) is an amino acid substitution. Preferably said substitution replaces a glycine amino acid residue with an arginine amino acid residue. Preferably said modification further comprises the deletion of at least 9, 10, 11, 12, 13 or 14 amino terminal amino acid residues of prolactin.

In a further preferred embodiment of the invention said chimeric polypeptide further comprises a binding domain of a cytokine receptor. Preferably said cytokine receptor is a growth hormone receptor.

In a preferred embodiment of the invention said binding domain comprises an extracellular binding domain of growth hormone receptor; preferably said domain consists of the extracellular domain of growth hormone receptor.

In an alternative preferred embodiment of the invention said receptor is a prolactin receptor.

In a preferred embodiment of the invention said binding domain comprises an extracellular binding domain of prolactin receptor; preferably said domain consists of the extracellular domain of prolactin receptor.

According to a further aspect of the invention there is provided a nucleic acid molecule that encodes a fusion or chimeric fusion polypeptide according to the invention.

According to an aspect of the invention there is provided a vector comprising a nucleic acid molecule according to the invention.

In a preferred embodiment of the invention said vector is adapted for the recombinant expression of said nucleic acid molecule.

A vector including nucleic acid (s) according to the invention need not include a promoter or other regulatory sequence, particularly if the vector is to be used to introduce the nucleic acid into cells for recombination into the genome for stable transfection.

Preferably the nucleic acid in the vector is operably linked to an appropriate promoter or other regulatory elements for transcription in a host cell. The vector may be a bi-functional expression vector which functions in multiple hosts.

By “promoter” is meant a nucleotide sequence upstream from the transcriptional initiation site and which contains all the regulatory regions required for transcription. Suitable promoters include constitutive, tissue-specific, inducible, developmental or other promoters for expression in eukaryotic or prokaryotic cells.

“Operably linked” means joined as part of the same nucleic acid molecule, suitably positioned and oriented for transcription to be initiated from the promoter. DNA operably linked to a promoter is “under transcriptional initiation regulation” of the promoter.

In a preferred embodiment the promoter is a constitutive, an inducible or regulatable promoter.

According to a further aspect of the invention there is provided a cell transfected or transformed with a nucleic acid molecule or vector according to the invention.

Preferably said cell is a eukaryotic cell. Alternatively said cell is a prokaryotic cell.

In a preferred embodiment of the invention said cell is selected from the group consisting of; a fungal cell (e.g. Pichia spp, Saccharomyces spp, Neurospora spp); insect cell (e.g. Spodoptera spp); a mammalian cell (e.g. COS cell, CHO cell); a plant cell.

According to a further aspect of the invention there is provided a method to manufacture a polypeptide according to the invention comprising:
i) providing a cell according to the invention;
ii) incubating said cell under conditions conducive to the production of said polypeptide; and optionally
iii) isolating said polypeptide from said cell or the growth media surrounding said cell.

In a preferred method of the invention said polypeptide is provided with an amino acid affinity tag to facilitate the isolation of said polypeptide.

Affinity tags are known in the art and include, maltose binding protein, glutathione S transferase, calmodulin binding protein and the engineering of polyhistidine tracts into proteins that are then purified by affinity purification on nickel containing matrices. In many cases commercially available vectors and/or kits can be used to fuse a protein of interest to a suitable affinity tag that is subsequently transfected into a host cell for expression and subsequent extraction and purification on an affinity matrix.

According to a further aspect of the invention there is provided a polypeptide according to the invention for use as a pharmaceutical.

According to a further aspect of the invention there is provided a nucleic acid according to the invention for use as a pharmaceutical.

According to a further aspect of the invention there is provided a pharmaceutical composition comprising a polypeptide according to the invention.

According to a yet further aspect of the invention there is provided a pharmaceutical composition comprising a nucleic acid molecule according to the invention. Preferably said nucleic acid molecule is part of a vector; preferably an expression vector adapted for eukaryotic expression.

In a preferred embodiment of the invention said pharmaceutical or pharmaceutical composition includes an excipient or carrier.

In a preferred embodiment of the invention said pharmaceutical or pharmaceutical composition is combined with a further therapeutic agent.

When administered the pharmaceuticals/compositions of the present invention is administered in pharmaceutically acceptable preparations. Such preparations may routinely contain pharmaceutically acceptable concentrations of salt, buffering agents, preservatives, compatible carriers, and optionally other therapeutic agents.

The pharmaceuticals/compositions of the invention can be administered by any conventional route, including injection. The administration and application may, for example, be oral, intravenous, intraperitoneal, intramuscular, intracavity, intra-articuar, subcutaneous, topical (eyes), dermal (e.g a cream lipid soluble insert into skin or mucus membrane), transdermal, or intranasal.

Pharmaceuticals/compositions of the invention are administered in effective amounts. An “effective amount” is that amount of pharmaceuticals/compositions that alone, or together with further doses or synergistic drugs, produces the desired response. This may involve only slowing the progression of the disease temporarily, although more preferably, it involves halting the progression of the disease permanently. This can be monitored by routine methods or can be monitored according to diagnostic methods.

The doses of the pharmaceuticals/compositions administered to a subject can be chosen in accordance with different parameters, in particular in accordance with the mode of administration used and the state of the subject (i.e. age, sex). When administered, the pharmaceuticals/compositions of the invention are applied in pharmaceutically-acceptable amounts and in pharmaceutically-acceptable compositions. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

The pharmaceuticals/compositions may be combined, if desired, with a pharmaceutically-acceptable carrier. The term “pharmaceutically-acceptable carrier” as used herein means one or more compatible solid or liquid fillers, diluents or encapsulating substances that are suitable for administration into a human. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction that would substantially impair the desired pharmaceutical efficacy.

The pharmaceuticals/compositions may contain suitable buffering agents, including: acetic acid in a salt; citric acid in a salt; boric acid in a salt; and phosphoric acid in a salt.

The pharmaceuticals/compositions also may contain, optionally, suitable preservatives, such as: benzalkonium chloride; chlorobutanol; parabens and thimerosal.

The pharmaceutical compositions may conveniently be presented in unit dosage form and may be prepared by any of the methods well-known in the art of pharmacy. All methods include the step of bringing the active agent into association with a carrier that constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as syrup, elixir or an emulsion.

Compositions suitable for parenteral administration conveniently comprise a sterile aqueous or non-aqueous preparation that is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

Polypeptides/nucleic acid molecules etc according to the invention can be incorporated into liposomes. Liposomes are lipid based vesicles which encapsulate a selected therapeutic agent which is then introduced into a patient. The liposome is manufactured either from pure phospholipid or a mixture of phospholipid and phosphoglyceride.

Typically liposomes can be manufactured with diameters of less than 200 nm; this enables them to be intravenously injected and able to pass through the pulmonary capillary bed. Furthermore the biochemical nature of liposomes confers permeability across blood vessel membranes to gain access to selected tissues. Liposomes do have a relatively short half-life. So called STEALTH® liposomes have been developed which comprise liposomes coated in polyethylene glycol (PEG). The PEG treated liposomes have a significantly increased half-life when administered intravenously to a patient. In addition, STEALTH® liposomes show reduced uptake in the reticuloendothelial system and enhanced accumulation selected tissues. In addition, so called immuno-liposomes have been develop which combine lipid based vesicles with an antibody or antibodies, to increase the specificity of the delivery of the agent to a selected cell/tissue.

The use of liposomes as delivery means is described in U.S. Pat. No. 5,580,575 and U.S. Pat. No. 5,542,935.

According to a further aspect of the invention there is provided the use of the polypeptide according to the invention in the manufacture of a medicament for the treatment of a condition selected from the group consisting of: gigantism, acromegaly; cancer (e.g. Wilm's tumour, osteogenic sarcoma, breast, colon, prostate, thyroid); diabetic retinopathy; diabetic nephropathy and other complications of diabetes and GH excess.

According to a further aspect of the invention there is provided a method of treatment of an animal, preferably a human, comprising administering an effective amount of a polypeptide according to the invention to said animal in need of treatment of a disease or condition that would benefit from inhibition of growth hormone or prolactin activity.

Examples of diseases that would benefit from the administration of the polypeptide antagonist would be apparent to the skilled person and would be any disease or condition that involves the activation or increased activation of growth hormone or prolactin receptor signal transduction.

In a preferred method of the invention said disease or condition is selected from the group consisting of: gigantism, acromegaly; cancer (e.g. Wilm's tumour, osteogenic sarcoma, breast, colon, prostate, thyroid); diabetic retinopathy; diabetic nephropathy and other complications of diabetes and GH excess.

As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “cancer” includes malignancies of the various organ systems, such as those affecting, for example, lung, breast, thyroid, lymphoid, gastrointestinal, and genito-urinary tract, as well as adenocarcinomas which include malignancies such as most colon cancers, renal-cell carcinoma, prostate cancer and/or testicular tumours, non-small cell carcinoma of the lung, cancer of the small intestine and cancer of the esophagus. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term “carcinoma” also includes carcinosarcomas, e.g., which include malignant tumours composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

According to a further aspect of the invention there is provided a method to modify the antagonist activity of a polypeptide according to the invention comprising the steps of:
i) providing a polypeptide encoded by a nucleic acid molecule selected from the group consisting of:
   a) a nucleic acid molecule consisting of the sequence as represented in FIG. 1 (SEQ ID NO: 1);
   b) a nucleic acid molecule comprising sequences that hybridise to the sequence identified in (a) wherein said nucleic acid molecule includes a modification comprising a sequence that encodes amino acid residue 176 wherein said modification results in the addition, substitution or deletion of at least one amino acid residue and said nucleic acid molecule encodes a polypeptide with growth hormone receptor antagonist activity
ii) mutating a codon that encodes a first amino acid residue of said polypeptide to produce a variant polypeptide;
iii) determining the inhibitory activity of the variant polypeptide with respect to growth hormone receptor activation thereby identifying a functional variant of said polypeptide.

According to a further aspect of the invention there is provided a variant polypeptide antagonist obtained or obtainable by the method according to the invention.

According to a further aspect of the invention there is provided a method for the rational design of mutations in a polypeptide comprising the steps of:
i) providing a 3D model of a first polypeptide as represented by the amino acid sequence in FIG. 2 (SEQ ID NO: 2);
ii) providing a 3D model of a variant polypeptide wherein said variant polypeptide is a modified sequence variant of said first polypeptide which is modified by addition, deletion or substitution of at least one amino acid residue in FIG. 2 (SEQ ID NO: 2);
iii) comparing the effect of the mutation on the 3D model of said second polypeptide when compared to the 3D model of said first polypeptide; and optionally
iv) testing the effect of said modification on the growth hormone receptor activation of said second polypeptide when compared to said first polypeptide.

According to a further aspect of the invention there is provided a homodimer comprising polypeptides comprising first and second polypeptides wherein said polypeptides comprise a first part that includes a polypeptide according to the invention, linked either directly or indirectly, to a second part wherein said second part comprises the extracellular domain of growth hormone receptor.

In a preferred embodiment of the invention said first part comprises the amino acid sequence as represented in FIG. 2a (SEQ ID NO: 2) wherein said amino acid sequence is modified by addition, deletion or substitution of at least one amino acid residue at position 176 and said second part comprising the extracellular domain of growth hormone receptor as represented by the amino acid sequence in FIG. 2b (SEQ ID NO: 4), 2c (SEQ ID NO: 5) or 2d (SEQ ID NO: 6).

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following figures

FIG. 1 (SEQ ID NO: 1) is the nucleic acid sequence of growth hormone circular permutation GHCP07;

FIG. 2a (SEQ ID NO: 2) is the amino acid sequence of growth hormone circular permutation GHCP07; FIG. 2b (SEQ ID NO: 4) is the amino acid sequence of the extracellular domain of growth hormone receptor; FIG. 2c (SEQ ID NO: 5) is the amino acid sequence of the A domain of growth hormone receptor; FIG. 2d (SEQ ID NO: 6) is the amino acid sequence of the B domain of growth hormone receptor.

FIG. 3 is the amino acid sequence of human prolactin (SEQ ID NO: 7);

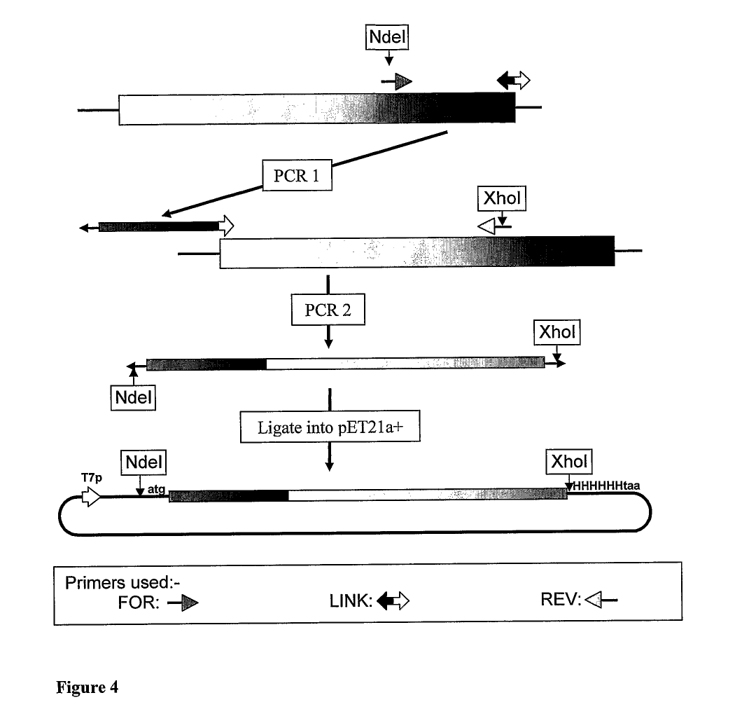

FIG. 4 is the strategy used to circularly permutate growth hormone;

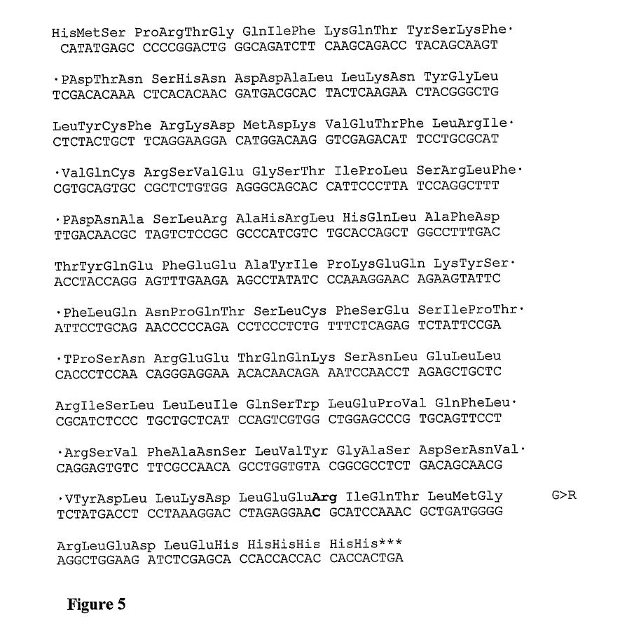

FIG. 5 (SEQ ID NO: 8) the nucleotide and amino acid (3 letter amino acid code) sequences of GHCP07BHis. The binding site 2 mutation is shown in bold. The amino acid change achieved by the mutation is shown to the right of the sequence (using 1 letter amino acid code);

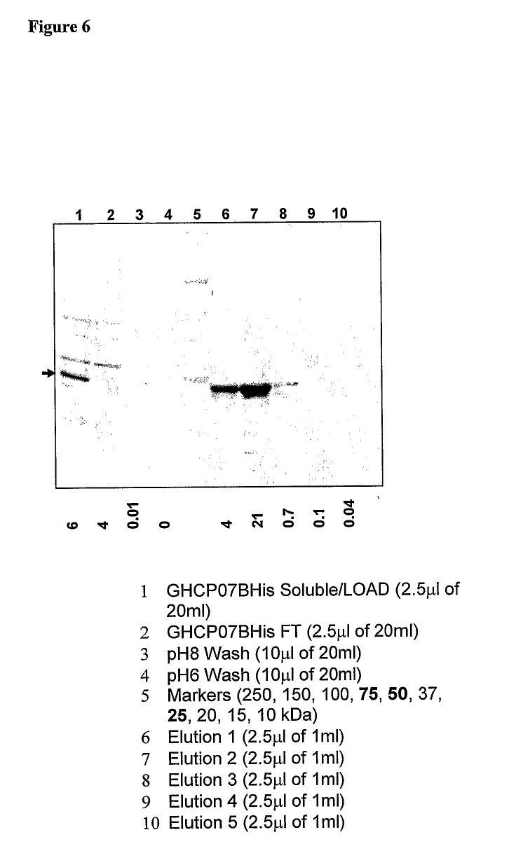

FIG. 6 SDS-PAGE gel showing the purification of GHCP07BHis; the contents of the lanes are shown below the gel and the protein concentration, in mg/ml, as measured by Bradfords assay is shown below each well;

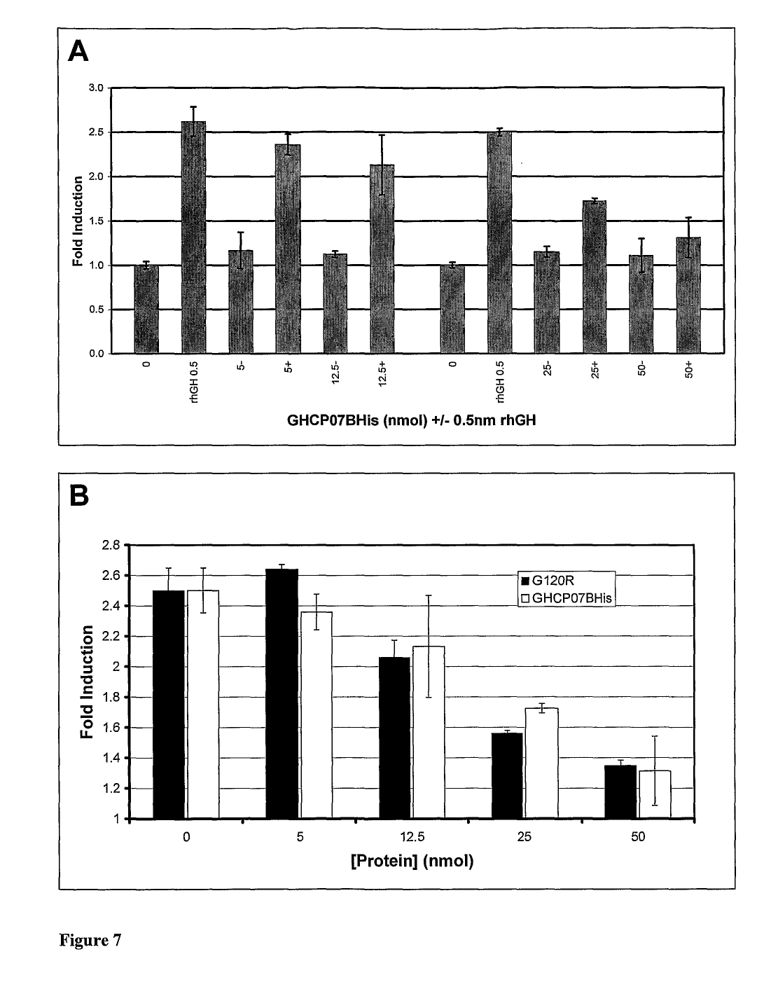

FIG. 7 A) Bioassay of GHCP07BHis showing its dose response in the absence and presence of 0.5 nmol rhGH. GHCP07BHis has no activity by itself and it antagonises the effect of rhGH. B) Comparison of the antagonistic activity of GHCP07BHis against GH.G120R. The activities of GHCP07BHis and GH.G120R are similar;

FIG. 8 (SEQ ID NO: 9): The nucleotide and amino acid (3 letter amino acid code) sequences of GHCP07CHis. The binding site 1 mutations are shown underlined and the binding site 2 mutation is shown in bold. The amino acid changes achieved by the mutations are shown to the right of the sequence (using 1 letter amino acid code);

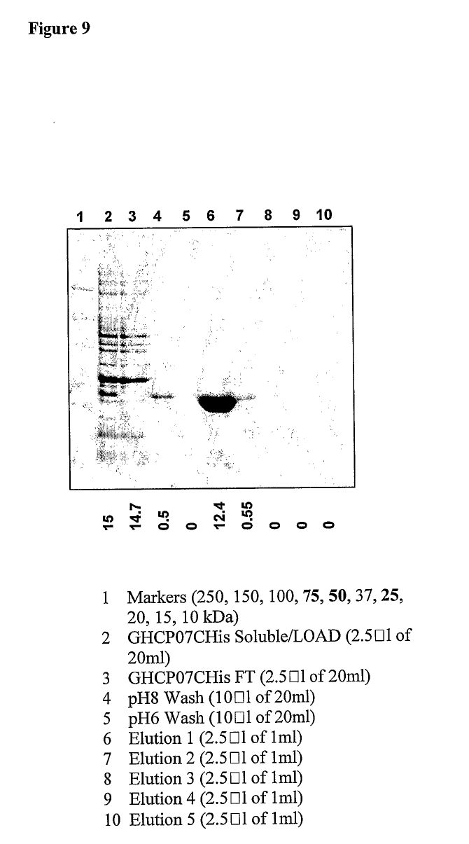

FIG. 9 SDS-PAGE gel showing the purification of GHCP07CHis; the contents of the lanes are shown below the gel and the protein concentration, in mg/ml, as measured by Bradfords assay is shown below each well; and

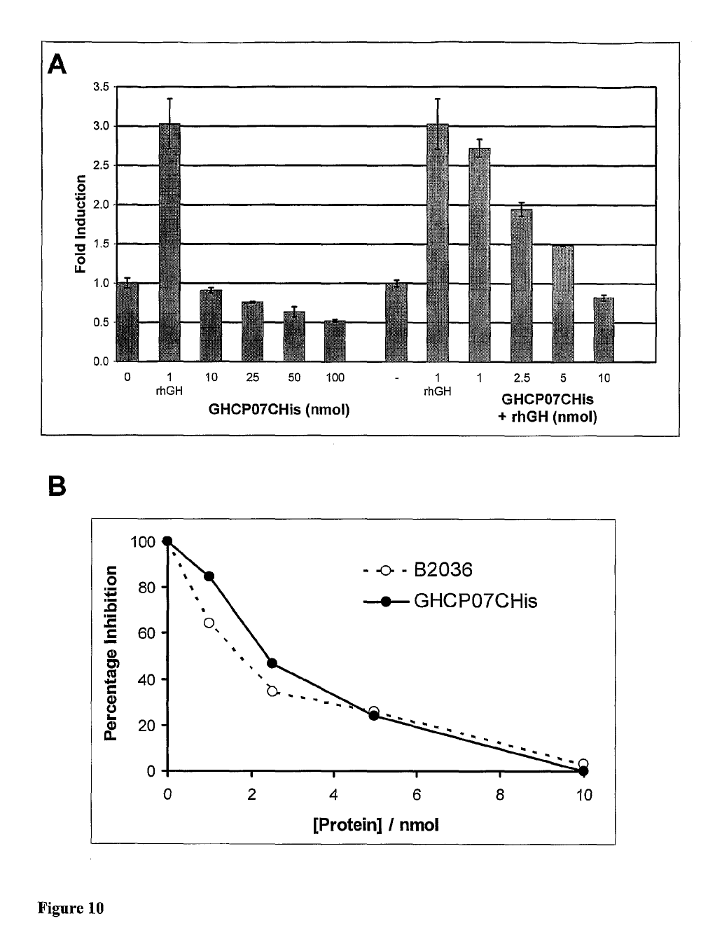

FIG. 10 A) Bioassay of GHCP07CHis showing its dose response in the absence and presence of 1 nmol rhGH. GHCP07CHis has no activity by itself and it antagonises the effect of rhGH. B) Comparison of the antagonistic activity of GHCP07CHis against B2036. The activities of GHCP07CHis and B2036 are similar.

### DEFINITIONS

Nucleic acid molecule: A nucleotide is a monomer that includes a base linked to a sugar, such as a pyrimidine, purine or synthetic analogs thereof, which when linked together form a nucleic acid molecule. A nucleic acid sequence refers to the sequence of bases in a nucleic acid molecule.

Polypeptide: A polymer in which the monomers are amino acid residues which are joined together through amide bonds. The terms “polypeptide” or “protein” as used herein are intended to encompass any amino acid sequence and include modified sequences such as glycoproteins. The term “polypeptide” is specifically intended to cover naturally occurring proteins, as well as those which are recombinantly or synthetically produced.

Variant polypeptide: A variant, i.e. a polypeptide and reference polypeptide may differ in amino acid sequence by one or more substitutions, additions, deletions, truncations which may be present in any combination. Among preferred variants are those that vary from a reference polypeptide by conservative amino acid substitutions. Such substitutions are those that substitute a given amino acid by another amino acid of like characters. The following non-limiting list of amino acids are considered conservative replacements (similar): a) alanine, serine, and threonine; b) glutamic acid and asparatic acid; c) asparagine and glutamine d) arginine and lysine; e) isoleucine, leucine, methionine and valine and f) phenylalaine, tyrosine and tryptophan. Most highly preferred are variants which retain the same biological function and activity as the reference polypeptide from which it varies. In addition, the invention features polypeptide sequences having at least 75% identity with the polypeptide sequences illustrated in FIG. 2, or fragments and functionally equivalent polypeptides thereof. In one embodiment, the polypeptides have at least 85% identity, more preferably at least 90% identity, even more preferably at least 95% identity, still more preferably at least 97% identity, and most preferably at least 99% identity with the amino acid sequences illustrated in FIG. 2.

Recombinant nucleic acid: A recombinant nucleic acid is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques. Similarly, a recombinant protein is one encoded by a recombinant nucleic acid molecule.

Fusion polypeptide: the translational fusion of at least two polypeptides to form single polypeptide typically manufactured as a recombinant polypeptide.

Peptide linker: a typically short peptide that can be helical and therefore provide a rigid connection between linked polypeptides or flexible and therefore provide a degree of rotational movement between linked polypeptides; or a combination of helical and non-helical to provide some rotational movement between linked polypeptides. Whilst the provision of an inflexible helical region maintains the spatial separation of the domains the provision of a flexible non-helical region enables the domains to orientate into the binding sites of the cytokine receptor(s). A peptide is typically a short polymer of amino acid residues.

Therapeutic agent: This is used in a generic sense and it includes treating agents, prophylactic agents, and replacement agents, for example agents that augment or enhance the therapeutic effect of a condition that would benefit from the administration of the polypeptides of the invention, for example immunomodulatory agents or chemotherapeutic agents.

Extracellular binding domain: refers to a part of a cell surface receptor that contacts a ligand to effect receptor mediated signal transduction. For example, the extracellular domain of growth hormone receptor exists as two linked domains each of approximately 100 amino acids, the C-terminal SD-100 (B domain) being closest to the cell surface and the N-terminal SD-100 domain (A domain) is being furthest away. It is a conformational change in these two domains that occurs on growth hormone or prolactin binding with the formation of the trimeric complex. Internalisation of the complex is followed by a recycling step whereby the receptor molecule is regenerated for further use within the cell.

### Materials and Methods

The circular permutation antagonist was synthesised using a two PCR strategy (FIG. 4); the template for the PCR was growth hormone which had been mutated in binding site 2 (G120R) or growth hormone that had been mutated in both binding site 1 and 2 (H18D, H21N, G120R, R167N, K168A, D171S, K172R, E174S and I179T). The primers FOR, LINK and REV used in the PCR reactions were GEPermLink- (5′-tggataagggaatggtgctgccctccacagag-3′ SEQ ID NO: 10), Nde-GHCP07F (5′-aattaattcatatgagcccccggactgggcag-3′ SEQ ID NO: 11) and GHCP07-XhoR (5′-aattctcgagatcttccagcctccccatc-3′ SEQ ID NO: 12), respectively. The PCR reactions were carried out using the EXPAND PCR kit (Roche) and the accompanying instructions were followed; the annealing temperatures for the first and second PCRs were 55° C. and 45° C., respectively. The final PCR product was ligated into pET21a+ (Novagen) between the NdeI and XhoI sites. The ligated plasmid was then transformed into chemically competent E. coli XL1 Blue cells. Plasmids made from colonies generated by the transformation were initially checked by restriction analysis using NdeI and XhoI. Clones which produced positive results in the restriction analysis was submitted for sequencing using T7 promoter and T7 terminator sequencing primers.

A single plasmid, with the correct sequence, was chosen and transformed into chemically competent E. coli BL21 (DE3). A colony of E. coli BL21 (DE3) transformed with the plasmid was picked and used to inoculate 20 ml LB media supplemented with carbenicillin (100 μg/ml). After an overnight incubation shaking at 37° C. the culture was used to provide a 2% inoculum for 500 ml LB supplemented with carbenicillin (100 μg/ml); this was then grown shaking at room temperature. When the OD600 of the culture reached ˜0.4 the culture was induced with IPTG, 1 mM final concentration, and then left shaking overnight at room temperature. The culture was then centrifuged to pellet the cells and the supernatant discarded.

The cell pellet was resuspended in 15 ml Equilibration buffer (20 mM Phosphate Buffer, 0.5M NaCl, 20% glycerol, 20 mM imidazole, and pH8) and then the cells lysed using a lysozyme/sodium deoxycholate/sonication treatment. The lysed cells were centrifuged at high speed to pellet the insoluble components and the supernatant then decanted to a fresh tube. The supernatant was made up to 20 ml using Equilibration buffer and then passed through a 0.2 μm syringe filter to further clarify the sample.

The His-tagged protein was purified using immobilised metal ion chromatography, Probond Resin (Invitrogen) charged with Ni²⁺ was used. 1 ml of resin was loaded into a column and equilibrated with 10 column volumes (CV) of Equilibration buffer. The clarified protein sample was then loaded onto the column. The column was washed with Equilibration buffer for 20CV and then with Wash buffer (20 mM Phosphate Buffer, 0.5M NaCl, 20% glycerol, pH6) until the A280 of the eluant was below 0.01. Bound protein was then eluted off the column using Elution buffer (20 mM Phosphate Buffer, 0.5M NaCl, 20% glycerol, 0.5M imidazole, pH6), six 1 ml fractions were collected. The eluted fractions were checked for content by SDS-PAGE gel analysis and by Bradfords protein assay.

Purified protein was submitted to the GH bioassay; agonistic activity was tested for by looking at stimulation by the test protein alone and antagonistic activity was tested for by looking at the activity of GH in the presence of the test protein.

### EXAMPLE

Circularly permutated growth hormone antagonist, GHCP07B (GHCP07 with the site 2 mutation), was generated by two PCR reactions, the first reaction produced a ˜200 bp product and this was used as a ‘megaprimer’ in a second PCR reaction to produce the circularly permutated growth hormone antagonist (GHCP07B) gene of ˜600 bp. The GHCP07B gene DNA fragment was digested NdeI and XhoI and then ligated into pET21a+ which had been digested by the same restriction enzymes. Transformation of this into E. coli XL1 Blue cells gave ˜500 colonies, with no colonies appearing on the negative control (transformed with water only) plate.

Three clones were picked for further processing; plasmid minipreps were made from these clones and the plasmid analysed by restriction analysis, all three clones gave the correct digestion pattern. These plasmids were then sequenced and the resulting sequence compared to the desired sequence (FIG. 5); two out of the three plasmids gave the correct sequence. One of these plasmids was then chosen to express and purify GHCP07BHis.

The plasmid was transformed into E. coli BL21 (DE3) and cultured. The resulting cells were lysed and His-tagged protein purified from the soluble fraction using a Ni-chelate column. The eluted protein was analysed by SDS-PAGE and Bradfords Protein Assay (FIG. 6); a total of ˜25 mg of protein was purified to >90% pure.

Elution 3 of the purification was used in the bioassay and a dose range of the GHCP07BHis activity was measured on its own and also in the presence of 0.5 nmol rhGH. This showed that GHCP07BHis had no agonistic activity and that it did have antagonistic activity (FIG. 7A). The activity of GHCP07BHis was comparable to that of GH.G120R (FIG. 7B).

Circularly permutated growth hormone antagonist, GHCP07C (GHCP07 with the site 1 and site 2 mutations), was generated and analysed in the same way as GHCP07B. The sequence of GHCP07C is shown in FIG. 8; the purification of the protein is shown in FIG. 9. Elution 1 of the purified protein was used in the bioassay. This showed that GHCP07C had no agonistic activity and was a potent antagonist (FIG. 10A) with activity comparable to B2036 (growth hormone with both the site 1 and site 2 mutations) (FIG. 10B).

1. A nucleic acid molecule comprising a sequence as represented in SEQ ID NO: 1 that encodes a polypeptide as represented in SEQ ID NO: 2 wherein the amino acid sequence is modified to include an amino acid addition, deletion or substitution of amino acid residue 176.

2. A nucleic acid molecule comprising a sequence as represented in SEQ ID NO: 1 that encodes a polypeptide as represented in SEQ ID NO: 2.

3. A nucleic acid molecule according to claim 1 that encodes a polypeptide comprising an amino acid sequence as represented in SEQ ID NO: 9.

4. A nucleic acid molecule according to claim 1 wherein said molecule encodes a polypeptide growth hormone antagonist.

5. A polypeptide comprising the amino acid sequence represented in SEQ ID NO: 2, which sequence has been modified by addition, deletion or substitution of at least one amino acid residue wherein said modification includes amino acid residue 176 and wherein said polypeptide is a growth hormone receptor antagonist.

6. A polypeptide according to claim 5 wherein said polypeptide is modified by substitution of glycine at position 176 with an amino acid selected from the group consisting of: histidine, aspartic acid, valine, arginine, alanine, lysine, tryptophan, tyrosine, phenylalanine and glutamic acid.

7. A polypeptide according to claim 6 wherein arginine or lysine or alanine are substituted for glycine residue 176.

8. A polypeptide according to claim 7 wherein said modification is glycine for arginine.

9. A polypeptide according to claim 5 wherein said polypeptide is represented by the amino acid sequence in SEQ ID NO: 9.

10. A polypeptide according to claim 5 wherein said polypeptide is linked to a second polypeptide comprising the extracellular binding domain of growth hormone receptor.

11. A polypeptide according to claim 10 wherein said second polypeptide consists of the extracellular domain of growth hormone receptor.

12. A polypeptide according to claim 11 wherein said second polypeptide consists of the amino acid sequence as represented in SEQ ID NO: 4.

13. A polypeptide according to claim 11 wherein said extracellular domain is the A domain of the extracellular domain of growth hormone receptor consisting of the amino acid sequence as represented in SEQ ID NO: 5.

14. A polypeptide according to claim 11 wherein said extracellular domain is the B domain of the extracellular domain of growth hormone receptor consisting of the amino acid sequence as represented in SEQ ID NO: 6.

15. A fusion polypeptide comprising at least two polypeptides according to claim 5 linked in tandem.

16. A fusion polypeptide according to claim 15 wherein said fusion polypeptide consists of two polypeptides linked in tandem.

17. A fusion polypeptide comprising a plurality of polypeptides according to claim 5.

18. A fusion polypeptide according to claim 10 wherein said polypeptides are linked together by a peptide linker molecule.

19. A fusion polypeptide according to claim 18 wherein said peptide linking molecule is a flexible peptide linker.

20. A fusion polypeptide according to claim 18 wherein the linker is a peptide which consists of 5 to 30 amino acid residues.

21. A fusion polypeptide according to claim 20 wherein the peptide linker consists of 10 to 20 amino acid residues.

22. A fusion polypeptide according to claim 18 wherein the linker comprises at least one copy of the peptide:
Gly-Gly-Gly-Gly-Ser (referred to as Gly4Ser) (SEQ ID NO: 3).

23. A fusion polypeptide according to claim 22 wherein the peptide linker is 10 amino acids in length and comprises two copies of the Gly4Ser.

24. A fusion polypeptide according to claim 22 wherein the peptide linker is 15 amino acids in length and comprises three copies of the Gly4Ser.

25. A fusion polypeptide according to claim 22 wherein the peptide linker is 20 amino acids in length and comprises four copies of the Gly4Ser linker.

26. A fusion polypeptide comprising at least two polypeptides according to claim 5 wherein said polypeptide further comprises at least one extracellular binding domain of growth hormone receptor.

27. A fusion polypeptide consisting of two polypeptides according to claim 5 and one extracellular binding domain of growth hormone receptor.

28. A chimeric fusion polypeptide comprising a polypeptide according to claim 5 linked, either directly or indirectly, to a prolactin polypeptide.

29. A chimeric fusion polypeptide according to claim 28 wherein said prolactin polypeptide comprises an amino acid sequence wherein said amino acid sequence is modified at position 129 of human prolactin as represented in SEQ ID NO 7, or an equivalent amino acid in an alternative prolactin polypeptide.

30. A chimeric fusion polypeptide according to claim 29 wherein said modification at position 129 as represented in SEQ ID NO: 7 is an amino acid substitution.

31. A chimeric fusion polypeptide according to claim 30 wherein said substitution replaces a glycine amino acid residue with an arginine amino acid residue.

32. A chimeric fusion polypeptide according to claim 28 wherein said prolactin polypeptide further comprises the deletion of at least 9, 10, 11, 12, 13 or 14 amino terminal amino acid residues.

33. A chimeric fusion polypeptide according to claim 29 wherein said polypeptide further comprises a ligand binding domain of a cytokine receptor.

34. A chimeric fusion polypeptide according to claim 33 wherein said cytokine receptor comprises an extracellular binding domain of growth hormone receptor.

35. A chimeric fusion polypeptide according to claim 34 wherein said cytokine receptor comprises an extracellular binding domain of prolactin receptor.

36. A chimeric fusion polypeptide according to claim 34 wherein said cytokine receptor consists of the extracellular domain of growth hormone receptor.

37. A chimeric fusion polypeptide according to claim 35 wherein said cytokine receptor consists of the extracellular domain of prolactin receptor.

38. A nucleic acid molecule that encodes a fusion or chimeric polypeptide according to claim 10.

39. A vector comprising a nucleic acid molecule according to claim 1.

40. A vector according to claim 39 wherein said vector is adapted for the recombinant expression of said nucleic acid molecule.

41. A cell transfected with a nucleic acid molecule according to claim 1.

42. A cell transformed with a nucleic acid molecule according to claim 1.

43. A cell according to claim 41 wherein said cell is a eukaryotic cell.

44. A cell according to claim 42 wherein said cell is a prokaryotic cell.

45. A method to manufacture a polypeptide comprising:
i) providing a cell according to claim 41;
ii) incubating said cell under conditions conducive to the production of said polypeptide; and optionally
iii) isolating said polypeptide from said cell or the growth media surrounding said cell.

46. A method according to claim 45 wherein said polypeptide is provided with an amino acid affinity tag to facilitate the isolation of said polypeptide.

47-48. (canceled)

49. A pharmaceutical composition comprising a polypeptide according to claim 3 and an excipient or carrier.

50. A pharmaceutical composition comprising a nucleic acid molecule according to claim 1 and an excipient or carrier.

51. A composition according to claim 50 wherein said nucleic acid molecule is part of a vector.

52. A composition according to claim 51 wherein said vector is an expression vector adapted for eukaryotic expression.

53. A composition according to claim 49 wherein said composition is combined with a further therapeutic agent.

54. (canceled)

55. A method of treatment of an animal comprising administering an effective amount of a polypeptide according to claim 5 to said animal in need of treatment of a disease or condition that would benefit from inhibition of growth hormone or prolactin activity.

56. A method according to claim 55 wherein said disease or condition is selected from the group consisting of: gigantism, acromegaly, cancer; diabetic retinopathy, diabetic nephropathy and other complications of diabetes and GH excess.

57. A method to modify the antagonist activity of a polypeptide comprising the steps of:
i) providing a polypeptide encoded by a nucleic acid molecule comprising a nucleic acid sequence as represented in SEQ ID NO: 1; and
ii) mutating a codon that encodes a first amino acid residue of said polypeptide to produce a variant polypeptide.

58. A variant polypeptide antagonist obtained or obtainable by the method according to claim 57.

59. A method for the rational design of mutations in a polypeptide comprising the steps of:
i) providing a 3D model of a first polypeptide as represented by the amino acid sequence in SEQ ID NO: 2;
ii) providing a 3D model of a variant polypeptide wherein said variant polypeptide is a modified sequence variant of said first polypeptide which is modified by addition, deletion or substitution of at least one amino acid residue as represented in SEQ ID NO: 2;
iii) comparing the effect of the mutation on the 3D model of said second polypeptide when compared to the 3D model of said first polypeptide; and optionally; and
iv) testing the effect of said modification on growth hormone receptor activation by the second polypeptide when compared to the first polypeptide.

60. A homodimer comprising two polypeptides according to claim 10.
